(19) United States
(12) Patent Application Publication
(10) Pub. No.: US 2008/0031908 A1
(43) Pub. Date: Feb. 7, 2008

(54) OILY COSMETIC COMPOSITION IN AEROSOL FORM

(75) Inventors: Odile AUBRUN-SONNEVILLE, Antony (FR); Dominique BORDEAUX, Soisy Sur Seine (FR)

Correspondence Address: OBLON, SPIVAK, MCCLELLAND MAIER & NEUSTADT, P.C., 1940 DUKE STREET, ALEXANDRIA, VA, 22314, US

(73) Assignee: L'OREAL, Paris (FR)

(21) Appl. No.: 11/782,697

(22) Filed: Jul. 25, 2007

Related U.S. Application Data
(60) Provisional application No. 60/834,177, filed on Jul. 31, 2006.

(30) Foreign Application Priority Data
Jul. 25, 2006 (FR) 06 53094

(51) Int. Cl. A61K 8/02; A61Q 1/14; A61Q 17/04; A61Q 19/00; A61Q 5/00

(52) U.S. Cl. 424/401; 424/47

(57) ABSTRACT

Aerosol product containing an oily composition containing at least one oil, and at least one hydrocarbon compound having a melting point greater than or equal to 30° C., the compound being in the form of solid particles and one or more propellants. The hydrocarbon compound can notably be a paraffin or a fatty acid amide. Used for cleaning and/or make-up removal of the skin and/or of the hair, care of the skin and/or of the hair, for protecting the skin against the sun, and for make-up of the skin.

### REFERENCE TO PRIOR APPLICATIONS

This application claims priority to U.S. provisional application 60/834,177 filed Jul. 31, 2006, and to French patent application 0653094 filed Jul. 25, 2006, both incorporated herein by reference.

### FIELD OF THE INVENTION

The invention relates to an aerosol product comprising an oily cosmetic composition that is able to produce a stable foam, containing a compound that is in the form of solid particles, and the use of said product in the form of aerosol for cleaning and/or make-up removal of the skin and/or of the hair, for the care of the skin and/or of the hair, for the protection of the skin against the sun, and for the make-up of the skin.

Additional advantages and other features of the present invention will be set forth in part in the description that follows and in part will become apparent to those having ordinary skill in the art upon examination of the following or may be learned from the practice of the present invention. The advantages of the present invention may be realized and obtained as particularly pointed out in the appended claims. As will be realized, the present invention is capable of other and different embodiments, and its several details are capable of modifications in various obvious respects, all without departing from the present invention. The description is to be regarded as illustrative in nature, and not as restrictive.

### BACKGROUND OF THE INVENTION

Oils are used extensively in the field of cosmetics, some of them for their effectiveness in make-up removal, others for their nutrient and protective properties for the skin or the hair. These oils are very often formulated as emulsions. However, to maximize their effects both for make-up removal and for nutrition, they can constitute a cosmetic aid on their own, for example when they are used as rinsable make-up removal oils, as body oils for the treatment of dry skin, as hair oils for the treatment of dry hair, as make-up products, or as sun oils.

Their application to the skin is generally not very easy, owing to their fluidity, which makes them difficult to handle. Moreover, the greasy sensation that they produce is often viewed as a disadvantage. It can therefore be advantageous to present them in the form of foam, as this provides a structured product that is easier to handle, and the sensory perception is greatly improved because the product is softer, since it is in the form of foam at the time of application, minimizing the perception of oiliness.

Oils that produce a foam can be obtained by pressurization of oil with a propellant and packaging as an aerosol. The problem is to obtain foams that are fine, while presenting sufficient stability.

Thus, document EP-A-1 438 946 describes aerosol compositions for generating an oil foam, comprising a grease or an oil and a fatty ester of polyglycerol. Document JP-A-04/89424 describes a composition in the form of aerosol comprising an oil, a zeolite, a surfactant, a foaming agent and a propellant. Document JP-A-09/110636 describes an oil-based foam-forming cleaning composition comprising a propellant and a liquid comprising oils, a fatty ester of dextrin, and a non-ionic surfactant. Document US-A-2005/186147 describes a foam-forming composition containing solid particles of metal oxide, of silica or of metals, a hydrophobic solvent, water, a surfactant, a stabilizer/gelling agent, and a propellant. However, the foams described according to these documents prove to be coarse and/or transient, “transient” signifying a time of stability of the foam of less than 1 minute.

### SUMMARY OF THE INVENTION

There is therefore a need for an oily foam-forming composition in aerosol form, which is comfortable and which gives a stable foam, i.e. a foam which lasts preferably for at least 5 minutes and better still for at least 10 minutes.

Now, it has been discovered, surprisingly, that hydrocarbon compounds present in the form of solid particles in the oily composition achieved the desired aim and provide an oily composition which, when used in an aerosol, gives a fine, stable foam, which is pleasant to use.

### DETAILED DESCRIPTION OF THE PREFERRED EMBODIMENTS

The present invention therefore relates to an aerosol product preferably useful for topical application comprising:

(a) an oily composition comprising at least one oil and at least one hydrocarbon compound having a melting point greater than or equal to 30° C., said hydrocarbon compound being in the form of solid particles, the quantity of hydrocarbon compound(s) ranging from 0.5 to 15 wt. % relative to the total weight of the composition, the amount of water and water-soluble compounds being less than 15 wt. % relative to the total weight of the oily composition, and

(b) one or more propellants.

“Aerosol product” means a product packaged in an aerosol device, suitable for pressurization. This device generally comprises a pressurized or pressurizable bottle equipped with a distributing head, and it is able to distribute a foam when the distributing head is actuated. Also making up a part of the invention is the oily composition itself, and this composition located in an aerosol device but with no propellant.

The aerosol product according to the invention is preferably intended for topical application and therefore preferably contains a physiologically acceptable medium. By physiologically acceptable medium, we mean a medium that is compatible with the skin, the mucous membranes (including the inside of the eyelids, the lips), the nails, the region around the eyes and/or keratin fibres (hair and eyelashes). This product preferably constitutes a cosmetic or dermatological composition.

The oily composition (a) is preferably anhydrous, i.e. free from water and water-soluble compounds such as polyols and C₂-C₆ monohydric alcohols. However, it can contain water and water-soluble compounds provided that the total amount of water and water-soluble compounds is at most 15 wt. % and preferably at most 10 wt. % relative to the total weight of the composition. The total content of water and water-soluble compounds can therefore range from 0 to 15% and preferably from 0 to 10% of the total weight of the composition. “Polyols” means water-soluble compounds containing at least two hydroxyl functions, for example glycerin, propylene glycol, butylene glycol, isoprene glycol and PEG-8. “C₂-C₆ monohydric alcohols” means compounds containing a single hydroxyl function, for example ethanol or isopropanol. Preferably, the oily composition is free from water.

The aerosol product can contain an amount of oily composition that is not limited, ranging for example from 80 to 97 wt. %, preferably from 80 to 95 wt. % and better still from 85 to 95 wt. %, relative to the total weight of the product.

The oily composition used in the aerosol foams as it leaves the aerosol, and it can therefore also be called a foam-forming composition, on the basis that it forms a foam when it is used in an aerosol containing a propellant.

The foam obtained as it leaves the aerosol according to the invention offers the advantage that it is fine and stable. While not bound or limited by theory, it is believed that this stability is due to the presence of the solid particles of hydrocarbon compound in the composition packaged in the aerosol.

The invention further relates to a foam obtained by spraying of the aerosol product as defined above.

Hydrocarbon Compounds

“Hydrocarbon compound” means a compound comprising mainly atoms of carbon and hydrogen. These compounds are not polymers, as the latter are defined as compounds containing the same unit several times and having a high molecular weight, whereas the hydrocarbon compounds according to the invention are non-polymeric compounds and they generally have a molecular weight below 1000, and preferably below 500.

According to a particular embodiment of the invention, the hydrocarbon compound used contains at least one heteroatom. This heteroatom or these heteroatoms present in this compound can notably be nitrogen and/or oxygen. The hydrocarbon compound can therefore contain one or more nitrogen atoms or one or more oxygen atoms or nitrogen atoms and oxygen atoms simultaneously.

The hydrocarbon compounds used in the composition of the invention preferably have a melting point greater than or equal to 30° C. and are in the form of solid particles in the composition of the invention, i.e. in the composition which is at a temperature below their melting point, they are not dissolved in the oils but they remain in solid form. However, they are hydrophobic compounds, which are soluble in the oils at a temperature above their melting point. The hydrocarbon compounds used preferably have a melting point ranging from 30° C. to 200° C., preferably from 30° C. to 150° C., and better still from 30° C. to 100° C.

The hydrocarbon compounds useful herein include:
paraffins;
fatty alcohols which contain oxygen as heteroatom;
fatty acids which contain oxygen as heteroatom;
fatty acid amides which contain oxygen and nitrogen as heteroatoms;
   and mixtures of these compounds.

“Fatty acid” means, in the present application, acids having at least 12 carbon atoms, preferably from 12 to 30 carbon atoms, more particularly from 14 to 22 carbon atoms, and “fatty alcohol” means alcohols having from 12 to 30 carbon atoms, more particularly from 14 to 22 carbon atoms.

As fatty alcohol, we may preferably mention for example cetyl alcohol, but it preferably refers to C₃₀-C₅₀ alcohols, such as the product marketed by the company New Phase Technologies under the designation Performacol 550-L-Alcohol (INCI name: C30-C50 Alcohols).

As fatty acid, we may preferably mention for example palmitic acid.

As paraffin, we may preferably mention, more particularly, refined paraffin, notably that marketed under the designation Cerafine 56/58 by the company Sasol.

As amides of fatty acids, we may preferably mention the amides derived from an acid having at least 12 carbon atoms. These amides are therefore acid amides having at least 12 carbon atoms, notably acid amides having from 12 to 30 carbon atoms, more particularly from 14 to 22 carbon atoms. The term “amide” includes the amides and their derivatives, for example derivatives having one or more hydroxyl functions, for example ethanolamides (monoethanolamides=MEA, or diethanolamides=DEA), mono-isopropanolamides (MIPA), and any other amide derivative.

Also useful herein are the following amides (INCI names):
Cocamide MEA (monoethanolamide of copra acids),
Cocamide MIPA (monoisopropanolamide of copra acids),
Cocamide DEA (diethanolamide of copra acids),
Stearamide MEA (monoethanolamide of stearic acid),
Hydroxystearamide MEA (N-(2-hydroxyethyl)-12-hydroxystearamide),
Myristamide MEA (monoethanolamide of myristic acid);
Lauramide MEA (monoethanolamide of lauric acid),
Palmitamide MEA (monoethanolamide of palmitic acid),
Myristoyl/Palmitoyl Oxostearamide/Arachamide MEA,
Cetyl-PG Hydroxyethyl Decanamide,
Cetyl-PG Hydroxyethyl Palmitamide,
Dibutyl Lauroyl Glutamide,
Ethylene Distearamide,
Hydroxycetyl Hydroxyethylstearamide,
PEG-4 Stearamide.
and mixtures thereof.

Preferably, the hydrocarbon compounds are selected from paraffins, C₃₀-C₅₀ fatty alcohols, fatty acid amides, and mixtures thereof. More preferably, they are selected from acid amides having at least 12 carbon atoms, notably the amides mentioned above.

The hydrocarbon compounds used according to the invention can be present in any amount, preferably an amount ranging from 0.5 to 15 wt. %, more preferably from 1 to 15 wt. %, even more preferably from 1 to 10 wt. % and better still from 2 to 10 wt. % relative to the total weight of the oily composition, including 2, 3, 4, 5, 6, 7, 8, 9, 10, 11, 12, 13, and 14 wt. % and all values and subranges between stated values.

Oils

The oily composition of the invention comprises one or more oils, and the amount of oils is not limited and can range for example from 60 to 99 wt. %, preferably from 65 to 98 wt. %, more preferably from 70 to 98 wt. % and better still from 75 to 98 wt. % relative to the total weight of the oily composition. By “oil”, we mean a fat that is liquid at room temperature (20 to 25° C.).

As oils that can be used in the composition of the invention, included are for example:
hydrocarbon oils of animal origin, such as perhydrosqualene;
hydrocarbon oils of vegetable origin, such as liquid triglycerides of fatty acids having from 4 to 22 carbon atoms such as triglycerides of heptanoic or octanoic acids or alternatively, for example, sweet almond oil, maize oil, soya oil, cucurbit oil, coriander oil, grape-seed oil, sesame oil, hazelnut oil, apricot oil, macadamia oil, arara oil, sunflower oil, castor oil, avocado oil, triglycerides of caprylic/capric acids such as those sold by the company Stearineries Dubois or those sold under the designations Miglyol 810, 812 and 818 by the company Dynamit Nobel, jojoba oil, shea butter oil;
synthetic esters and ethers, notably of fatty acids, such as the oils of formulae R¹COOR² and R¹OR²in which R¹ represents a fatty acid residue having from 8 to 29 carbon atoms, and R² represents a linear or branched hydrocarbon chain, containing from 3 to 30 carbon atoms, for example purcelline oil, isononyl isononanoate, isopropyl myristate, isopropyl palmitate, ethyl-2-hexyl palmitate (or octyl palmitate), octyl-2-dodecyl stearate, octyl-2-dodecyl erucate, isostearyl isostearate; hydroxylated esters such as isostearyl lactate, octylhydroxystearate, octyldodecyl hydroxystearate, diisostearyl malate, triisocetyl citrate, heptanoates, octanoates, decanoates of fatty alcohols; polyol esters, such as dioctanoate of propylene glycol, neopentylglycol diheptanoate and diethyleneglycol diisononanoate ; esters of pentaerythritol such as pentaerythrityl tetraisostearate; ester of benzoic acid and of C₁₂-C₁₅ alcohols (INCI name: C12-15 Alkyl Benzoate);
linear or branched hydrocarbons with melting point below 30° C., of mineral or synthetic origin, such as volatile or non-volatile paraffin oils, and their derivatives, petroleum jelly, isohexadecane, isododecane, hydrogenated polyisobutene such as oil of Parleam®; cyclic alkanes such as dioctyl cyclohexane;
fatty alcohols having from 8 to 26 carbon atoms, with melting point below 30° C., such as octyldodecanol, 2-butyloctanol, 2-hexyldecanol, 2-undecylpentadecanol, oleic alcohol or linoleic alcohol;
fluorinated oils partially hydrocarbon-containing and/or silicone-containing such as those described in document JP-A-2-295912. As fluorinated oils, we may also mention perfluoromethylcyclopentane and perfluoro-1,3-dimethylcyclohexane, sold under the designations “FLUTEC PC1®” and “FLUTEC PC3®” by the company BNFL Fluorochemicals; perfluoro-1,2-dimethylcyclobutane; perfluoroalkanes such as dodecafluoropentane and tetradecafluorohexane, sold under the designations “PF 5050®” and “PF 5060®” by the company 3M, or also bromoperfluorooctyl sold under the designation “FORALKYL®” by the company Atochem; nonafluoromethoxybutane sold under the designation “MSX 4518®” by the company 3M and nonafluoroethoxyisobutane; derivatives of perfluoromorpholine, such as 4-trifluoromethyl perfluoromorpholine sold under the designation “PF 5052®” by the company 3M;
silicone oils such as volatile or non-volatile polymethylsiloxanes (PDMS) with a linear or cyclic silicone chain, liquid or pasty at room temperature, notably cyclopolydimethylsiloxanes (cyclomethicones) such as cyclohexasiloxane; polydimethylsiloxanes with alkyl, alkoxy or phenyl groups, pendent or at the end of the silicone chain, groups having from 2 to 24 carbon atoms; phenylated silicones such as phenyltrimethicones, phenyldimethicones, phenyltrimethylsiloxydiphenyl-siloxanes, diphenyl-dimethicones, diphenylmethyidiphenyl trisiloxanes, 2-phenylethyltrinmethyl-siloxysilicates, and polymethylphenylsiloxanes;
mixtures thereof.

In the list of oils mentioned above, “hydrocarbon oil” means any oil having mostly carbon and hydrogen atoms, and optionally ester, ether, fluoro groups, carboxylic acid, alcohol, silicone, amine, phenyl, and/or amino acid.

Additives

The oily composition of the product of the invention can also contain various additives, which can be soluble in the oils, and/or can be dispersed in the oily medium.

Thus, it can contain for example one or more surfactants that are soluble or dispersible in the oils.

As surfactants of this type, included are for example:
oxyethylene fatty acid and polyol esters, such as oxyethylenated glyceryl triisostearate (20 OE) (INCI name: PEG-20 glyceryl triisostearate), sorbitan trioleate 20 OE (INCI name: Polysorbate 85); oxyethylenated fatty acid esters and their derivatives such as polyethylene glycol mono-isostearate (8 OE) (INCI name: PEG-8 isostearate) and copolymer of polyethylene glycol (30 OE) and of 12-hydroxystearic acid (Arlacel P135 from the company Uniqema; INCI name: PEG-30 Dipolyhydroxystearate);
silicone-containing surfactants such as alkyl dimethicone copolyols, notably those having an alkyl radical with 10 to 22 carbon atoms and having 2 to 50 oxyethylene groups and 2 to 50 oxypropylene groups, such as cetyl dimethicone copolyol (INCI name: Cetyl PEG/PPG-10/1 Dimethicone) such as the product marketed under the designation Abil EM-90 by the company Goldschmidt; lauryl dimethicone copolyol (INCI name: Lauryl PEG/PPG-18/18 Methicone) and for example a mixture of about 91% lauryl dimethicone copolyol and about 9% isostearyl alcohol, marketed under the designation DOW CORNING 5200 FORMULATION AID by the company Dow Corning; dimethicone copolyols, notably among those having 2 to 50 oxyethylene groups and 2 to 50 oxypropylene groups, for example that having 18 oxyethylene groups and 18 oxypropylene groups (PEG/PPG-18/18 Dimethicone), such as the mixture of dimethicone copolyol having 18 oxyethylene groups and 18 oxypropylene groups, cyclopentasiloxane and water (10/88/2), marketed by the company Dow Corning under the designation DC-3225C or DC2-5225C (INCI name: Cyclopentasiloxane/PEG/PPG-18/18 Dimethicone), or that having 14 oxyethylene groups and 14 oxypropylene groups (PEG/PPG-14/14 Dimethicone) such as, notably, the mixture of dimethicone copolyol having 14 oxyethylene groups and 14 oxypropylene groups and cyclopentasiloxane (85/15) marketed under the designation Abil EM-97 by the company Goldschmidt (INCI name: Bis-PEG/PPG-14/14 Dimethicone/Cyclopentasiloxane);
the alkyl polyglycosides (APG) such as decyl glycoside, for instance the product marketed under the designation MYDOL 10® by the company Kao Chemicals, the product marketed under the designation PLANTAREN 2000 UP® by the company Cognis, and the product marketed under the designation ORAMIX NS 10® by the company Seppic; the caprylyl/capryl glycoside such as the product marketed under the designation ORAMIX CG 110® by the Company Seppic; the lauryl glycoside for instance the products marketed under the designations PLANTAREN 1200 N® and PLANTACARE 1200® by the company Cognis; and the coco-glycoside such as the product marketed under the designation PLANTACARE 818/UP® by the company Cognis;
and mixtures of these surfactants.

When they are present, the quantity of surfactant(s) is not limited and can range for example from 0.5 to 30 wt. %, preferably from 1 to 20 wt. % and better still from 5 to 20 wt. % relative to the total weight of the oily phase.

According to a preferred embodiment of the invention, the oily composition (a) contains at least one surfactant, preferably a non-ionic surfactant selected from the fatty acid and polyol oxyethylene esters.

The oily composition of the invention can also contain particles, for example pigments such as titanium dioxide, oxides of zirconium, cerium, zinc or iron (black, yellow or red); clays such as disteardimonium hectorite; silicas; particles containing acrylic copolymers (INCI name: Acrylates Copolymer) such as micro-spheres: expanded vinylidene chloride/acrylonitrile/PMMA, marketed under the designations Expancel; starch particles; and mixtures thereof.

The oily composition of the invention can also contain cosmetic actives, for example antioxidants, keratolytic and anti-ageing agents such as alpha-hydroxyacids (lactic acid, glycolic acid, citric acid) and beta-hydroxyacids (salicylic acid and derivatives such as N-alkyl salicylic acids, for example N-octanoyl-5 salicylic acid); vitamins such as vitamin E (tocopherol and derivatives), vitamin C (ascorbic acid and derivatives), vitamin A (retinol and derivatives); emollients; organic or inorganic UV filters; and any active usually employed according to the intended use of the composition.

Furthermore, the oily composition of the invention can contain lipophilic polymers such as the block copolymers derived from styrene such as the styrene/ethylene-butylene/styrene copolymer such as the product marketed under the designation Kraton G-1650E by the company Kraton Polymers; the copolymers of acrylic or methacrylic acid, such as the acrylate/stearyl acrylate/dimethicone methacrylate copolymer marketed under the designation KP 561 P by the company Shin Etsu; the poly-C10-30-alkyl acrylates such as the product marketed under the designation Interlimer IPA 13-1 by the company Landec; the ethylenediamine/stearyl dimer dilinoleate copolymers, such as the product marketed under the designation Uniclear 100 VG by the company Arizona Chemical.

As stated above, the oily composition of the invention can also contain hydrophilic solvents such as water, polyols and C₂-C₆ monohydric alcohols, in a maximum amount of 15 wt. % and preferably 10 wt. % relative to the total weight of the oily composition. However, it is preferably water-free.

The presence of alcohol such as ethanol may be useful, to provide better reconstitution of the product by liquefying the juices, which undergo considerable gelation owing to the presence of the hydrophobic compounds of the invention. It also makes it possible to improve the stability of some compositions which have a tendency to thicken over time. Thus, the composition can contain alcohol and notably ethanol in an amount ranging for example from 0.01 to 10 wt. %, preferably from 0.05 to 10 wt. % and more preferably from 0.05 to 5 wt. % relative to the total weight of the oily composition.

Propellant

The aerosol product according to the invention comprises the oily composition described above and one or more propellants. The propellants used in the product according to the invention are selected from liquefied gases, for example dimethyl ether (DME); volatile hydrocarbons such as n-butane, propane, isobutane, and mixtures thereof, optionally with at least one chlorinated and/or fluorinated hydrocarbon, for example the compounds sold by the company Dupont de Nemours under the designations Freon® and Dymel®, and in particular monofluorotrichloromethane, difluorodichloromethane, tetrafluorodichloroethane, and 1,1-difluoroethane sold notably under the trade name DYMEL 152 A by the company DUPONT. We may optionally add a compressed gas such as carbon dioxide, nitrous oxide, nitrogen and/or air.

The amount of liquefied gas is not limited and can range for example from 5 to 20 wt. % and preferably from 5 to 15 wt. % relative to the total weight of the aerosol product.

The aerosol product according to the invention is placed in packaging that is suitable for aerosols.

The pressure in the aerosol can vary in any amount, for example from 1 to 10 bar and preferably from 1.5 to 7 bar. The compressed gas or gases which can be added to the liquefied gas make it possible to reach a higher pressure than if the liquefied gas were the only propellant, and, when they are present, the compressed gases are added in an amount sufficient to provide the pressure that is required, for example a pressure of at least 3 bar and preferably of at least 5 bar.

The aerosol product according to the invention can be used in the field of cosmetics notably for cleaning and/or make-up removal of the skin and/or of the hair, for the care of the skin and/or of the hair, for the protection of the skin against the sun or UV, for the make-up of the skin, for example as a product for cleaning and/or make-up removal of the skin and/or of the hair, as a body oil for softening and nourishing the skin, as sun oil for protection against sunlight or UV radiation, or as hair oil for protecting the hair fibres, for nourishing them and to facilitate untangling, or as a make-up product for the eyelashes or the skin. The foam formed is fine and stable.

Thus, the present application further relates to the cosmetic use of the aerosol product as defined above, for cleaning and/or make-up removal of the skin and/or of the hair, for the care of the skin and/or of the hair, for the protection of the skin against the sun or UV radiation, and for make-up of the skin.

The oily compositions of the product according to the invention can be prepared by mixing the constituents at a temperature above or equal to the melting temperature of the solid hydrocarbon compounds, mixing preferably being carried out in a turbine mixer. Then the compositions are cooled while stirring until they have cooled to room temperature (about 20 to 25° C.). The solid hydrocarbon compounds solidify during cooling. The compositions may then be pressurized using one or more propellants, and optionally one or more liquefied gases.

The invention further relates to a method of preparation of the aerosol product as defined above, comprising preparing a composition as defined above, by mixing the constituents at a temperature above or equal to the melting temperature of the hydrocarbon compounds, cooling the composition, while stirring, until it has cooled to room temperature, and adding one or more propellants to the composition (one or more liquefied gases and optionally one or more compressed gases).

### EXAMPLES

The following examples are given to illustrate the invention and are not limiting. All the amounts are given as percentage by weight of raw material relative to the total weight of the composition.

### Examples 1 to 3 According to the Invention

| | Example 1 | Example 2 | Example 3 |
|---|---|---|---|
| Composition A | | | |
| (without gas) | | | |
| Ethylhexyl palmitate | 52.45 | 50.45 | 37.5 |
| Isononyl isononanoate | 28 | — | 36.5 |
| Isododecane | — | 28 | |
| Preservatives | 0.55 | 0.55 | |
| PEG-20 glyceryl | 16 | 8 | 16 |
| triisostearate (1) | | | |
| Cocamide MEA (2) | 3 | | |
| Cocamide MIPA (3) | — | 5 | |
| Paraffin (4) | — | — | 10 |
| Composition B = Aerosol | | | |
| (composition A + gas) | | | |
| Composition A | 90 | 90 | 90 |
| Dimethyl ether | — | 10 | — |
| Isobutane | 10 | — | 10 |
| Nitrogen at a pressure | Qsf up to 5 | Qsf up to 5 | Qsf up to 5 |
| of 5 bar | bar | bar | bar |
| Appearance of the juice | Gel of | Suspension | Suspension |
| | particles, which | of particles | of particles |
| | becomes fluid | | |
| | when stirred | | |
| Quality of the foam | Fine, stable | Fine, stable | Quite fine, |
| | foam | foam | stable foam |

(1) EMALEX GWIS-320EX from the company Ajinomoto
(2) COMPERLAN 100 from the company Cognis
(3) REWOMID V 3203 from the company Goldschmidt
(4) CERAFINE 56/58 PASTILLES from the company Sasol

Examples 1 and 3 can constitute, for example, products for cleaning the skin, and Example 2 can constitute for example a body oil.

### Comparative Examples 1 to 3

| | Comparative | Comparative | Comparative |
|---|---|---|---|
| | example 1 | example 2 | example 3 |
| Composition A | | | |
| (without gas) | | | |
| Ethylhexyl palmitate | 39.225 | 55.45 | — |
| Isononyl isononanoate | 39.225 | — | 75.45 |
| Isododecane | — | 28 | — |
| Preservatives | 0.55 | 0.55 | 0.55 |
| PEG-20 glyceryl | 16 | 8 | 16 |
| triisostearate (1) | | | |
| Dextrin palmitate (5) | 5 | — | — |
| Composition B = Aerosol | | | |
| (composition A + gas) | | | |
| Composition A | 90 | 90 | 90 |
| Dimethyl ether | 10 | — | 10 |
| Isobutane | — | 10 | — |
| Compressed nitrogen | Qsf up to 5 | — | — |
| at 5 bar | bar | | |
| Appearance of the juice | Thick solution | Clear | Clear |
| | with lumps | solution | solution |
| Quality of the foam | Fine foam, | Coarse, | Very coarse, |
| | quickly | unstable | unstable |
| | becoming | foam | foam |
| | unstable | | |

(1) EMALEX GWIS-320EX from the company Ajinomoto
(5) RHEOPEARL KL2 - OR from the company Chiba Flour Milling

These comparative examples show that, in the absence of a compound forming solid particles in the oil and with a melting point greater than or equal to 30° C., the juice can be heterogeneous (see comparative example 1) and the foam obtained is either fine but unstable (comparative example 1) or coarse and unstable (comparative examples 2 and 3).

### Comparative Examples 4 and 5

| | Comparative | Comparative |
|---|---|---|
| | example 4 | example 5 |
| Composition A | | |
| (without gas) | | |
| Ethylhexyl palmitate | 40 | 40 |
| Isononyl isononanoate | 39 | 39 |
| PEG-20 glyceryl | 16 | 16 |
| triisostearate (1) | | |
| Zeolite (6) | 5 | — |
| Polymethyl | — | 5 |
| Silsesquioxane (7) | | |
| Composition B = Aerosol | | |
| (composition A + gas) | | |
| Composition A | 90 | 90 |
| isobutane | 10 | 10 |
| Compressed nitrogen | Qsf up to 5 bar | Qsf up to 5 bar |
| at 5 bar | | |
| Appearance of the juice | Clear solution with | Clear solution with |
| | solid deposit at the | solid deposit at the |
| | bottom which does | bottom which does |
| | not disperse when | not disperse when |
| | stirred | stirred |
| Quality of the foam | Very unstable foam | Very unstable foam |

(1) EMALEX GWIS-320EX from the company Ajinomoto
(6) X-MOL from Zeochem
(7) TOSPEARL 145A from GE Toshiba Silicones

These comparative examples 4 and 5 show that replacement of the compound forming solid particles in the oil and having a melting point greater than or equal to 30° C., by solid polymer particles, gives a heterogeneous juice, and an unstable aerosol foam.

### Examples 4 to 6 According to the Invention

| | Example 4 | Example 5 | Example 6 |
|---|---|---|---|
| Composition A | | | |
| Ethylhexyl palmitate | 58.45 | 50.45 | 52.45 |
| Isononyl isononanoate | — | 28 | 28 |
| Isododecane | 28 | — | — |
| Preservatives | 0.55 | 0.55 | 0.55 |
| Ethanol | — | 2 | — |
| Polyglyceryl-4 | 8 | — | — |
| Isostearate (5) | | | |
| Polysorbate 85 (or | — | — | 16 |
| sorbitan trioleate | | | |
| 20 OE) (6) | | | |
| PEG-20 glyceryl | — | 16 | — |
| triisostearate (1) | | | |
| Cocamide MEA (2) | 2 | 3 | 3 |
| Cocamide MIPA (3) | 4 | — | — |
| Composition B = | | | |
| Aerosol | | | |
| (composition A + gas) | | | |
| Composition A | 90 | 90 | 90 |
| Isobutane | — | 10 | 10 |
| Dimethyl ether | 10 | — | — |
| Nitrogen at a pressure | — | — | Qsf up |
| of 5 bar | | | to 5 bar |
| Appearance of the juice | Solution | Solution | Gel of |
| | thickened with | thickened with | particles |
| | particles | particles | |
| Quality of the foam | Fine, stable | Fine, stable | Fine, stable |
| | foam | foam | foam |

(1) EMALEX GWIS-320EX from the company Ajinomoto
(2) COMPERLAN 100 from the company Cognis
(3) REWOMID V 3203 from the company Goldschmidt
(5) ISOLAN GI 34 from the company Goldschmidt
(6) RHEODOL TW - 0320 V from the company KAO

Example 4 can constitute for example a body oil, and Examples 5 and 6 can constitute for example products for cleaning the skin.

### Example 7 According to the Invention: Foundation

| | Example 7 |
|---|---|
| Composition A (without gas) | |
| Ethylhexyl palmitate | Qsp 100% |
| Isononyl isononanoate | 40 |
| Isododecane | — |
| Preservatives | 0.55 |
| PEG-20 glyceryl triisostearate (1) | 4 |
| Cocamide MEA (2) | 3 |
| Titanium dioxide (untreated anatase) | 6 |
| Brown and yellow iron oxides | 1 |
| Composition B = Aerosol | |
| (composition A + gas) | |
| Composition A | 90 |
| Isobutane | 10 |
| Nitrogen at a pressure of 5 bar | Qsf up to 5 bar |

(1) EMALEX GWIS-320EX from the company Ajinomoto
(2) COMPERLAN 100 from the company Cognis

### Example 8 According to the Invention: “Sun Oil” Composition

| | Example 8 |
|---|---|
| Composition A | |
| Ethylhexyl palmitate | Qsf 100% |
| Isododecane | 28 |
| Preservatives | 0.55 |
| PEG-20 glyceryl triisostearate (1) | 8 |
| Cocamide MEA (2) | 2 |
| Cocamide MIPA (3) | 4 |
| Butyl Methoxy Dibenzoylmethane (7) | 4 |
| Octocrylene (8) | 4 |
| Titanium dioxide (9) | 3.5 |
| Composition B = Aerosol | |
| (composition A + gas) | |
| Composition A | 90 |
| Dimethyl ether | 10 |

(1) EMALEX GWIS-320EX from the company Ajinomoto
(2) COMPERLAN 100 from the company Cognis
(3) REWOMID V 3203 from the company Goldschmidt
(7) PARSOL 1789 from the company DSM Nutritional Products
(8) UVINUL N539 from the company BASF
(9) MICRO TITANIUM DIOXIDE MT100 AQ from the company TAYCA

The above written description of the invention provides a manner and process of making and using it such that any person skilled in this art is enabled to make and use the same, this enablement being provided in particular for the subject matter of the appended claims, which make up a part of the original description and including an aerosol product suitable for topical application, comprising (a) an oily composition containing at least one oil and at least one hydrocarbon compound having a melting point greater than or equal to 30° C., said compound being in the form of solid particles, the quantity of hydrocarbon compound(s) ranging from 0.5 to 15 wt. % relative to the total weight of the composition, the amount of water and of water-soluble compounds being less than 15 wt. % relative to the total weight of the oily composition, and (b) one or more propellants.

As used herein, the phrases “selected from the group consisting of,” “chosen from,” and the like include mixtures of the specified materials. Terms such as “contain(s)” and the like as used herein are open terms meaning ‘including at least’ unless otherwise specifically noted. Phrases such as “mention may be made,” etc. preface examples of materials that can be used and do not limit the invention to the specific materials, etc., listed.

All references, patents, applications, tests, standards, documents, publications, brochures, texts, articles, etc. mentioned herein are incorporated herein by reference. Where a numerical limit or range is stated, the endpoints are included. Also, all values and subranges within a numerical limit or range are specifically included as if explicitly written out.

The above description is presented to enable a person skilled in the art to make and use the invention, and is provided in the context of a particular application and its requirements. Various modifications to the preferred embodiments will be readily apparent to those skilled in the art, and the generic principles defined herein may be applied to other embodiments and applications without departing from the spirit and scope of the invention. Thus, this invention is not intended to be limited to the embodiments shown, but is to be accorded the widest scope consistent with the principles and features disclosed herein. In this regard, certain embodiments within the invention may not show every benefit of the invention, considered broadly.

1. An aerosol product comprising:
(a) an oily composition comprising at least one oil and at least one hydrocarbon compound having a melting point greater than or equal to 30° C., said compound being in the form of solid particles, the quantity of hydrocarbon compound(s) being 0.5 to 15 wt. % relative to the total weight of the composition, the amount of water and of water-soluble compounds being less than 15 wt. % relative to the total weight of the oily composition, and
(b) one or more propellants.

2. The aerosol product according to claim 1, wherein the hydrocarbon compound contains at least one heteroatom.

3. The aerosol product according to claim 2, wherein the at least one heteroatom is nitrogen and/or oxygen.

4. The aerosol product according to claim 1, wherein the at least one hydrocarbon compound is selected from paraffins, fatty alcohols, fatty acids, fatty acid amides, and mixtures thereof.

5. The aerosol product according to claim 1, comprising at least one hydrocarbon compound that is an amide derivative of an acid having at least 12 carbon atoms.

6. The aerosol product according to claim 5, wherein the hydrocarbon compound is selected from Cocamide MEA, Cocamide MIPA, Cocamide DEA, stearamide MEA, Hydroxystearamide MEA, Myristamide MEA, Lauramide MEA, Palmitamide MEA, Myristoyl/palmitoyl oxostearamide/arachamide MEA, Cetyl-PG hydroxyethyl Decanamide, Cetyl-PG hydroxyethyl palmitamide, Dibutyl lauroyl glutamide, Ethylene Distearamide, Hydroxycetyl hydroxyethylstearamide, PEG-4 Stearamide, and mixtures thereof.

7. The aerosol product according to claim 4, comprising at least one fatty alcohol selected from the C₃₀ to C₅₀ fatty alcohols.

8. The aerosol product according to claim 1, comprising at least one refined paraffin.

9. The aerosol product according to claim 1, wherein the amount of oil(s) is 60 to 99 wt. % relative to the total weight of the oily composition.

10. The aerosol product according to claim 1, wherein the oily composition further comprises one or more surfactants that are soluble or dispersible in the oil(s).

11. The aerosol product according to claim 10, comprising at least one surfactant selected from fatty acid and polyol oxyethylene esters, fatty acid oxyethylene esters and their derivatives, silicone-containing surfactants, alkylpolyglycosides, and mixtures thereof.

12. The aerosol product according to claim 1, wherein the oily composition further comprises at least one C₂-C₆ monohydric alcohol.

13. The aerosol product according to claim 1, wherein the amount of oily composition is 80 to 97 wt. % relative to the total weight of the product.

14. The aerosol product according to claim 1, wherein the propellant comprises a liquefied gas selected from dimethyl ether, n-butane, propane, isobutane, and mixtures thereof.

15. The aerosol product according to claim 14, wherein the amount of liquefied gas is 5 to 20 wt. % relative to the total weight of the product.

16. The aerosol product according to claim 1, further comprising at least one compressed gas selected from carbon dioxide, nitrous oxide, nitrogen or air.

17. The aerosol product according to claim 1, pressurized at a pressure of 1 to 10 bar.

18. A method for cleaning and/or make-up removal of the skin and/or of the hair, care of the skin and/or of the hair, for protecting the skin against the sun, and for make-up of the skin, comprising applying the aerosol product according to claim 1 to the skin.

19. An oily composition comprising at least one oil and at least one hydrocarbon compound having a melting point greater than or equal to 30° C., said compound being in the form of solid particles, the quantity of hydrocarbon compound(s) being 0.5 to 15 wt. % relative to the total weight of the composition, the amount of water and of water-soluble compounds being less than 15 wt. % relative to the total weight of the oily composition.

20. The oily composition according to claim 19, wherein said oily composition is anhydrous.
